(19) United States
(12) Patent Application Publication
(10) Pub. No.: US 2015/0305917 A1
(43) Pub. Date: Oct. 29, 2015

(54) Urine Collector

(71) Applicant: Sophia Hai Yun SU, North Point (HK)

(72) Inventor: Sophia Hai Yun SU, North Point (HK)

(21) Appl. No.: 14/673,784

(22) Filed: Mar. 30, 2015

Related U.S. Application Data
(63) Continuation-in-part of application No. 14/670,399, filed on Mar. 26, 2015.

(30) Foreign Application Priority Data
Apr. 29, 2014 (HK) 14104119.2
Jun. 11, 2014 (CN) 201420309147.4

(51) Int. Cl. A61F 5/451

(52) U.S. Cl. CPC A61F 5/451

(57) ABSTRACT

A urine collector for collecting urine from a user includes a collector, a top cover coupled on top of the collector, and a urine guider. The collector has a bottom wall and a surrounding wall upwardly extended therefrom to define a cavity. The top cover has a ring shape and defines a top side with a size adapted for covering a hip of the user, wherein the top side of the top cover is sloped downwardly from an outer peripheral edge to an inner peripheral edge thereof for guiding urine flowing into the collector. The urine guider is provided at a bottom portion of the collector for guiding urine at the urine guider within the cavity of the collector and then for guiding the urine to a container. The urine collector is light in weight, easy to use, and able to prevent the overflow of urine in the collector.

### CROSS REFERENCE OF RELATED APPLICATION

This is a Continuation-In-Part application that claims the benefit of priority under 35 U.S.C. §119 to a non-provisional application, application Ser. No. 14/670,399, filed Mar. 26, 2015.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

The present invention relates to a health care product, and more particularly to a urine collector for bedridden female patients.

2. Description of Related Arts

The use of a urinary collection device is well known to collect urine from a physically-disabled person. Especially for female patients, the female urinary collection device can substantially reduce the workload for health care workers. Generally, a potty or a bed urinal is used as the urinary collection device to collect urine from the patient. To a physically-disabled patient, urination is a hardship to the patient or the patient family member or nurse who takes care the patient. Generally, such potty or bed urinal, which is relatively high to the patient, is used to collect urine from a physically-disabled person. It may require one or two people to assist the patient for each urination and it is really suffering to the patient. In other words, such conventional urinary collection devices are difficult to use for urine collection and urine always leaks between the contacting surface of the urinary collection device and the body of the patient. Bed linens and clothing are contaminated due to the urine leakage, so as to cause different health problems such as infection or pressure ulcers.

An improved urine collector is a simple, comfortable to wear, effective, and convenience appliance to take care of the bedridden patients, wherein the improved urine collector can reduce workloads for nurses and families. In other words, the conventional urine collector can help the care workers and/or patient's family members to reduce the stress for caring the bedridden patients.

China Patent Publication No. ZL20102019533939, ZL200720024857.2, ZL200420053761.5, and ZL200620081221.7 disclosed an improved urine collector for bedridden female patients to solve the common problems of the current urine collectors. Since the improved urine collector can only cover on the meatus urinarius of the female patient, the improved urine collector is difficult to collect all discharging urine from the patients. As a result, bed linens and clothing are easily to be contaminated by the urine. In addition, physically-disabled female patients with low limb fracture, pelvic fracture, or postpartum are difficult to use the above mentioned urine collectors because they are not powerful enough to retain the urine collector in position. An improper use of urine collector may cause serious injures due to the excessive movement of the patients.

### SUMMARY OF THE PRESENT INVENTION

An object of the present invention is to provide a urine collector, wherein the urine collector is formed in a compact structure and is light in weight, such that the urine collector can be used by the patients for overnight. In other words, health care workers do not need to frequently unload the urine in the urine collector of the present invention.

Another object of the present invention is to provide a urine collector which has a relatively thinner structure and can discharge urine continuously.

Another object of the present invention is to provide a urine collector which is adapted to collect urine throughout the hip area but not limiting to the urination portion, so as to prevent leaking out of urine. In addition, a top side a top cover of the collector of the urine collector is inclinedly extended from an outer peripheral edge toward a center, so as to ensure and guide all the urine be collected toward a container through a top opening of the collector to avoid splashing and leakage. Since the collector is not used to store urine being collected but to function as a transition means to continue to guide the container, the collector can be constructed in compact size with a thinner thickness and a lighter weight that enables an overnight usage without the need to replace the collector. Accordingly, to a less severe patient, the patient can place the collector in position by himself or herself and there is no need to have anyone to help for urination. To a more severe patient, it simply needs someone to slight support the hip of the patient to place the collector in position for urination that substantially alleviates suffering to the patient and reduces burden of the care for the patient. For hospitals or senior care facilities, the collector can be directly connected to the sewage drain system that links to a urine collection container for ease of filtration or disinfection treatment, so that the present invention can save a lot of manpower and meet the environmental health requirement.

According to the present invention, the foregoing and other objects and advantages are attained by a urine collector, comprising a collector, a transporting tube, and a container. The transporting tube can be a PET tube, a latex tube, or a silica gel tube. The collector is pot-shaped container having a top opening, and is preferably made of hard plastic, foam material, or hard rubber. The urine collector further comprises a ring-shaped top cover coupled on a top edge of the collector, wherein the top cover is preferably made of plastic or latex, and is obliquely extended toward a center of the top cover, such that a top side of the top cover is slanted and downwardly extended toward the top opening of the collector. The urine collector further comprises a guider and a discharging unit arranged on a bottom portion of the collector, wherein a top side of the guider is lower than a bottom surface of the collector. In addition, the collector has a surrounding sidewall, wherein the surrounding sidewall can be a straight wall, an obliquely and downward-extended arc-shaped wall, or an inclined straight wall. It is worth mentioning that the collector also can be made of metal or wood.

Accordingly, the discharging unit comprises a collecting port and a discharging port located on an inner wall of the transporting tube, wherein the collecting port is operatively communicated with the guider and the discharging port is operatively linked with the transporting tube. The collecting port and the discharging port are operatively connected with each other.

Accordingly, the hip portion of the patient sits on the collector that the collector will cover the urethra of the patient so as to prevent the leakage of the urine, wherein the collector serves as a temporary collector for guiding the urine flowing into the container, such that the urine collector can be used for urine without frequently discharging urine in the collector.

Accordingly, the container is not limited to a container with or without scale and a sewage drain and the like.

The present invention is simple in structure, light in weight, and easy to operate, such that the present invention has benefits for the bedridden patients for clinical care and home care, and it is also convenient for the bedridden patients to solve their urinating problems by themselves who cannot get to a restroom to urinate in a convenience and privacy manner. At the same time, the ring-shaped top cover is preferably made of latex, which is very comfortable for the user to sit on, and the ring-shaped top cover is slightly tilted towards a center thereof to ensure the discharging urine flowing into the collector. The shape of the guider and the surrounding sidewall are able to facilitate the discharging urine flowing into the collector, such that the urine can be effectively discharged from the collector to the container, so as to reduce the leakage of urine for contaminating the bedding, and further reduce the incidence of the ulcers.

Additional advantages and features of the invention will become apparent from the description which follows, and may be realized by means of the instrumentalities and combinations particular point out in the appended claims.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of a urine collector according to a preferred embodiment of the present invention.

FIG. 2 is a sectional view of the urine collector according to the above preferred embodiment of the present invention.

FIG. 3 illustrates a first alternative mode of the collector of the urine collector according to the above preferred embodiment of the present invention, illustrating the arc-shaped wall of the collector.

FIG. 4 illustrates a second alternative mode of the collector of the urine collector according to the above preferred embodiment of the present invention, illustrating the inclined wall of the collector.

FIG. 5 is a perspective view of a urine collector according to a second preferred embodiment of the present invention.

FIG. 6 is a sectional view of the urine collector according to the second preferred embodiment of the present invention.

FIGS. 7A to 7C illustrate different configurations of the urine directing grooves of the urine collector according to the second preferred embodiment of the present invention.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

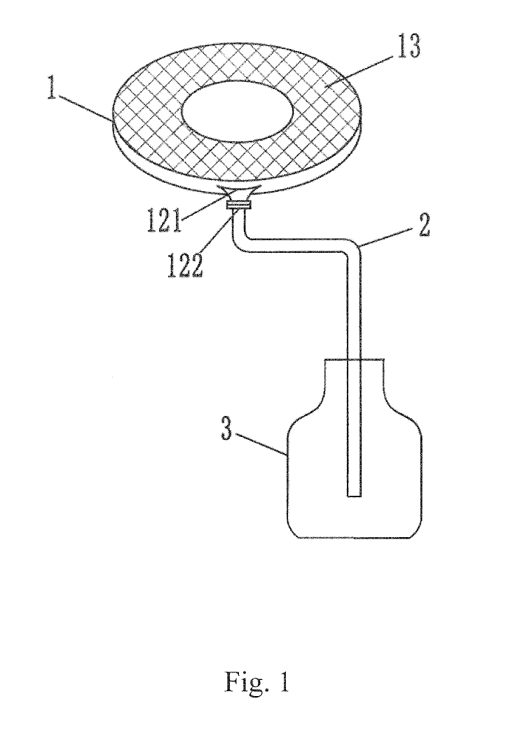

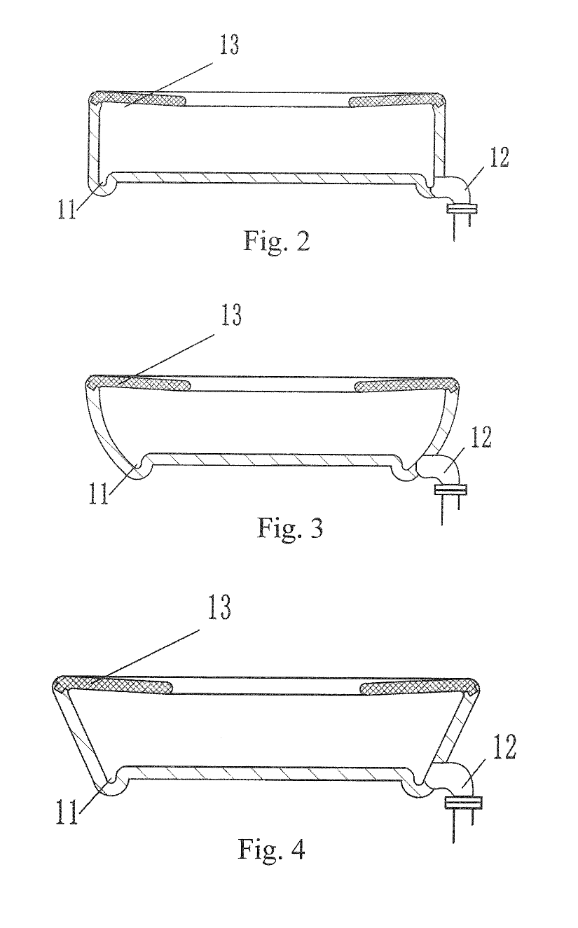

Referring to FIGS. 1 and 2 of the drawings, a urine collector according to a preferred embodiment of the present invention is illustrated, wherein the urine collector comprises a collector 1 having a top opening and a cavity, a transporting tube 2, which is made of PET material, connected to the collector 1 for transporting the urine, and a container 3 operatively connected with the transporting tube 2 for containing the urine from the collector 1 through the transporting tube 2. The collector 1 is a pot-shaped collector and made of a rigid plastic material. Accordingly, the collector 1 comprises a urine guider 11 provided at a bottom portion of the collector 1 for guiding urine at the urine guider 11 within the cavity, and a discharging unit 12 operatively connected with the urine guider 11 of the collector 1, wherein the urine guider 11 is a guiding groove indented around a peripheral portion of the bottom wall of the collector 1. It is worth mentioning that a top portion of the urine guider 11 is lower than the bottom wall of the collector 1. And, the collector 1 further comprises a surrounding wall, wherein the surrounding wall is a vertical wall extended with respect to the bottom wall of the collector 1 that the size of the top opening of the collector 1 matches the size of the bottom side of the collector 1. In other words, the urine guider 11 is formed between the surrounding wall and the bottom wall of the collector 1.

Moreover, the collector 1 further comprises a ring-shaped top cover 13 coaxially coupled on a top edge of the collector 1 to partially cover the top opening thereof, wherein the top cover 13 is preferably made of latex. A top side of the top cover 13 is inclinedly extended from an outer peripheral edge toward a center, so as to extend toward the top opening of the collector 1 to guide the urine toward the container 1 through the top opening thereof. In other words, the slope of the top side of the top cover 13 is gradually decreased from an outer edge of the top cover 13 to an inner edge thereof. Furthermore, the discharging unit 12 comprises a collecting port 121 located at the surrounding wall of the container 1 and a discharging port 122, wherein the collecting port 121 is operatively linked with the urine guider 11 and the discharging port 122 is operatively linked with the transporting tube 2, and the collecting port 121 and the discharging port 122 are operatively connected with each other. The discharging port 122 is connected to the inner wall of the transporting tube 2. In other words, the urine moves along the guider 11 to reach the collecting port 121, and passes through the collecting port 121 and the discharging port 122 respectively, so as to reach the transporting tube 2, and then the urine moves along the transporting tube 2 to be guide into the container 3.

Accordingly, the collector 1 is placed below a hip of the patient, especially female patient, wherein the collector serves as a temporary collecting station for guiding the urine to flow into the container 3.

FIG. 3 illustrates a first alternative mode the urine collector, wherein the urine collector comprises a collector 1 having a top opening and a cavity, a transporting tube 2, which is made of latex material, connected to the collector 1 for transporting the urine, and a container 3 operatively connected with the transporting tube 2 for containing the urine from the collector 1 through the transporting tube 2. The collector 1 is a pot-shaped collector and made of a rigid rubber material. Accordingly, the collector 1 comprises a urine guider 11 provided at a bottom portion of the collector 1 for guiding urine at the urine guider 11 within the cavity, and a discharging unit 12 operatively connected with the urine guider 11 of the collector 1, wherein the urine guider 11 is a guiding groove indented around a peripheral portion of the bottom wall of the collector 1. It is worth mentioning that a top portion of the urine guider 11 is lower than the bottom wall of the collector 1. And, the collector 1 further comprises a surrounding wall, wherein the surrounding wall is an arc-shaped wall extended with respect to the bottom wall of the collector 1 that the size of the top opening of the collector 1 is larger than the size of the bottom wall of the collector 1. In other words, the urine guider 11 is formed between the surrounding wall and the bottom wall of the collector 1.

Moreover, the collector 1 further comprises a ring-shaped top cover 13 coaxially coupled on a top edge of the collector 1 to partially cover the top opening thereof, wherein the top cover 13 is preferably made of latex. A top side of the top cover 13 is inclinedly extended from an outer peripheral edge toward a center, so as to extend toward the top opening of the collector 1 to guide the urine toward the container 1 through the top opening thereof. In other words, the slope of the top side of the top cover 13 is gradually decreased from an outer edge of the top cover 13 to an inner edge thereof. Furthermore, the discharging unit 12 comprises a collecting port 121 located at the surrounding wall of the container 1 and a discharging port 122, wherein the collecting port 121 is operatively linked with the urine guider 11 and the discharging port 122 is operatively linked with the transporting tube 2, and the collecting port 121 and the discharging port 122 are operatively connected with each other. The discharging port 122 is connected to the outer wall of the transporting tube 2. In other words, the urine moves along the guider 11 to reach the collecting port 121, and passes through the collecting port 121 and the discharging port 122 respectively, so as to reach the transporting tube 2, and then the urine moves along the transporting tube 2 to be guide into the container 3.

Accordingly, the collector 1 is placed below a hip of the patient, especially female patient, wherein the collector serves as a temporary collecting station for guiding the urine to flow into the container 3.

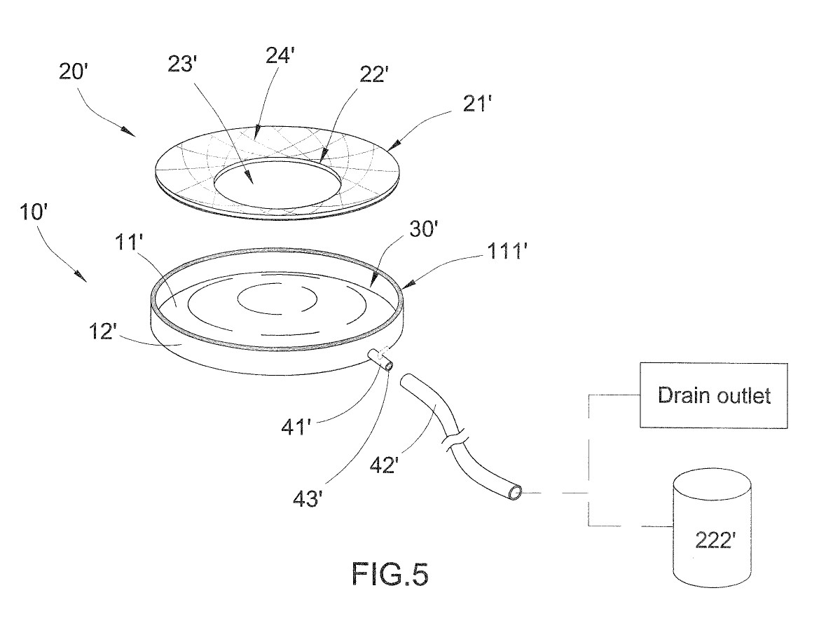

FIG. 4 illustrates a second alternative mode the urine collector, wherein the urine collector comprises a collector 1 having a top opening and a cavity, a transporting tube 2, which is made of polycarbonate material, connected to the collector 1 for transporting the urine, and a container 3 operatively connected with the transporting tube 2 for containing the urine from the collector 1 through the transporting tube 2. The collector 1 is a pot-shaped collector and made of a rigid rubber material. Accordingly, the collector 1 comprises a urine guider 11 provided at a bottom portion of the collector 1 for guiding urine at the urine guider 11 within the cavity, and a discharging unit 12 operatively connected with the urine guider 11 of the collector 1, wherein the urine guider 11 is a guiding groove indented around a peripheral portion of the bottom wall of the collector 1. It is worth mentioning that a top portion of the urine guider 11 is lower than the bottom wall of the collector 1. And, the collector 1 further comprises a surrounding wall, wherein the surrounding wall is an inclined wall extended with respect to the bottom wall of the collector 1 that the size of the container 1 is gradually reduced from the top opening of the collector 1 to the bottom wall of the collector 1. In other words, the urine guider 11 is formed between the surrounding wall and the bottom wall of the collector 1.

Moreover, the collector 1 further comprises a ring-shaped top cover 13 coaxially coupled on a top edge of the collector 1 to partially cover the top opening thereof, wherein the top cover 13 is preferably made of latex. A top side of the top cover 13 is inclinedly extended from an outer peripheral edge toward a center, so as to extend toward the top opening of the collector 1 to guide the urine toward the container 1 through the top opening thereof. In other words, the slope of the top side of the top cover 13 is gradually decreased from an outer edge of the top cover 13 to an inner edge thereof. Furthermore, the discharging unit 12 comprises a collecting port 121 located at the surrounding wall of the container 1 and a discharging port 122, wherein the collecting port 121 is operatively linked with the urine guider 11 and the discharging port 122 is operatively linked with the transporting tube 2, and the collecting port 121 and the discharging port 122 are operatively connected with each other. The discharging port 122 is connected to the outer wall of the transporting tube 2. In other words, the urine moves along the guider 11 to reach the collecting port 121, and passes through the collecting port 121 and the discharging port 122 respectively, so as to reach the transporting tube 2, and then the urine moves along the transporting tube 2 to be guide into the container 3.

Accordingly, the collector 1 is placed below a hip of the patient, especially female patient, wherein the collector serves as a temporary collecting station for guiding the urine to flow into the container 3.

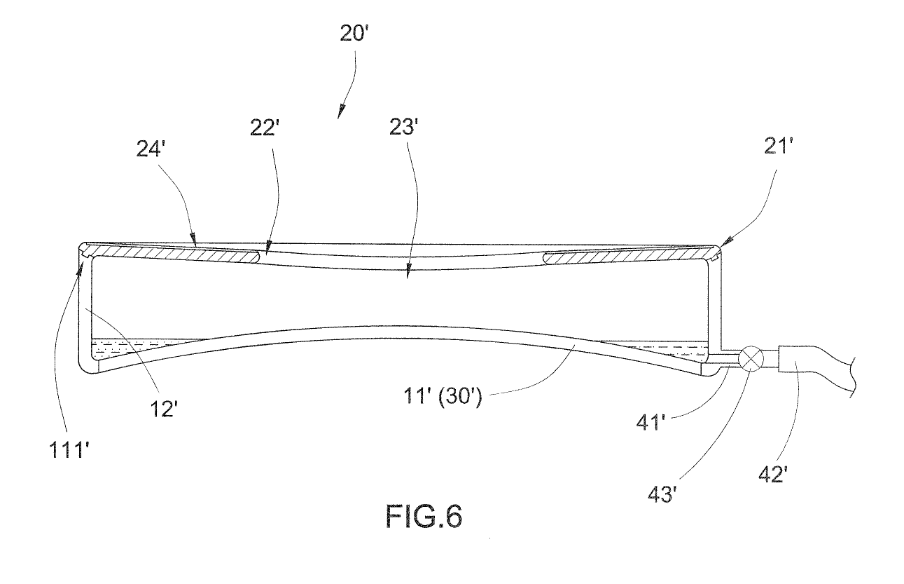

As shown in FIG. 5, a urine collector according to a second embodiment illustrates an alternative mode of the first embodiment, wherein the urine collector is arranged for collecting urine from a user, especially a female user. As shown in FIGS. 5 and 6, the urine collector comprises a basin which comprises a collector 10′ and a top cover 20′, and a urine guider 30′.

Accordingly, the basin has a cavity for collecting urine, a top side having a size adapted for covering a hip of the user, and a top opening formed at the top side to communicate with the cavity. The basin is arranged for being placed underneath the hip of the user, such that the top side of the basin is large enough to cover the urethra of the user to prevent the leakage of the urine. Preferably, the user is able to sit on the top side of the basin to collect the urine in the cavity through the top opening. In addition, the top opening of the basin is large enough to cover the urethra of the user.

The collector 10′ has a bottom wall 11′ and a surrounding wall 12′ upwardly extended from the bottom wall 11′ to define the cavity within the bottom wall 11′ and the surrounding wall 12′. Accordingly, the collector 10′ is a shallow pan configuration that the collector 10′ can be slid underneath the hip of the user. Preferably, the depth of the cavity, i.e. the height of the surrounding wall 12′, is about one inch deep. The collector 10′ is preferably configured to have a circular shape. It is appreciated that the collector 10′ can be formed in oval shape or irregular shape. The bottom wall 11′ of the collector 10′ can be a flat bottom wall.

The top cover 20′ is detachably supported on top of the collector 10′, wherein the top cover 20′ has a ring shape defining an outer peripheral edge 21′, an inner peripheral edge 22′, and a through hole 23′ within the inner peripheral edge 22′. The top opening of the basin is defined at the through hole 23′ of the top cover 20′ and the top side of the basin is defined at the top side of the top cover 20′.

Accordingly, the outer peripheral edge 21′ of the top cover 20′ is detachably coupled at the surrounding wall 12′ of the collector 10′. Preferably, a coupling slot 111′ is formed at a top edge of the surrounding wall 12′ of the collector 10′, wherein the outer peripheral edge 21′ of the top cover 20′ is detachably coupled at the coupling slot 111′ of the collector 10′. It is worth mentioning that the surrounding wall 12′ of the collector 10′ can be configured to have a vertical wall, an arc-shaped wall, or an inclined wall as mentioned in the first embodiment.

As shown in FIG. 6, the top side of the top cover 20′ is sloped downwardly from the outer peripheral edge 21′ of the top cover 20′ to the inner peripheral edge 22′ thereof for guiding the urine to flow into the collector 10′. In case the urine is spilled on the top cover 20′, the slanted top side of the top cover 20′ will guide the urine to flow toward the through hole 23′ so as to collect the urine in the collector 10′.

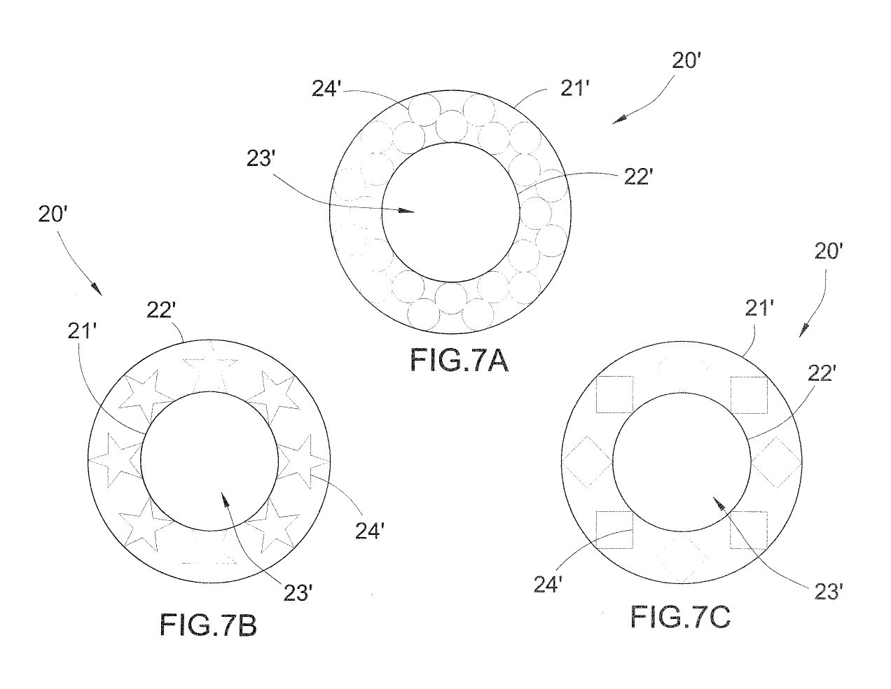

In addition, the top cover 20′ further has a plurality of urine directing grooves 24′ indently formed on the top side of the top cover 20′ and extended to the inner peripheral edge 22′ thereof for guiding the urine to flow toward the through hole 23′ of the top cover 20′. As shown in FIG. 5, the urine directing grooves 24′ are grouped into a first groove set and a second groove set, wherein the first and second groove sets are intersected with each other. Preferably, the urine directing grooves 24′ are elongated straight grooves radially extended toward the through hole 23′ of the top cover 20′. It is worth mentioning that the urine directing grooves 24′ can be configured to have different shapes and sizes, such as circular shape in FIG. 7A, star shape in FIG. 7B, or rectangular shape in FIG. 7C, on the top side of the top cover 20′ to guide the flow of urine into the cavity.

The urine guider 30′ is provided at a bottom portion of the basin for collecting urine at the urine guider 30′ within the cavity of the basin. In particular, the urine guider 30′ is provided at the bottom wall 11′ of the collector 10′ at a peripheral portion thereof for collecting the urine at the urine guider 30′ within the cavity of the basin so as to enhance a stabilization of the collector 10′ after the urine is collected. It is worth mentioning that when the urine is collected in the cavity of the collector 10′, the urine will be moved by the movement of the collector 10′. Therefore, the center of mass of the basin will be shifted, which may cause the basin to be flipped unintentionally. When the urine is collected at the urine guider 30′ around the peripheral portion of the bottom wall 11′ of the collector 10′, the urine will be retained at the peripheral portion of the collector 10′ by the weight force, such that the collector 10′ can be stabilized to minimize the shifting of the center of mass due to the movement of the collector 10′.

As shown in FIG. 6, the urine guider 30′ is integrated with the bottom wall 11′ of the collector 10′ that the bottom wall 11′ is a convex wall radially sloping down toward the peripheral portion for collecting the urine at the peripheral portion of the collector 10′ so as to enhance a stabilization of the collector 10′ after the urine is collected. It is appreciated that the urine guider 30′ can be configured to have a guiding groove indentedly formed at the peripheral portion of the bottom wall 11′ of the collector 10′, as mentioned in the first embodiment, for collecting the urine at the peripheral portion of the collector 10′ so as to enhance a stabilization of the collector 10′ after the urine is collected.

The urine collector further comprises a discharging unit 40′ operatively connected with the urine guider 30′ for discharging the urine therefrom, wherein the discharging unit 40′ comprises a discharging port 41′ formed at the basin to communicate with the urine guider 30′ and a transporting tube 42′ detachably coupled at the discharging port 41′ for discharging the urine at the urine guider 30′ through the discharging port 41′.

As shown in FIG. 6, the discharging port 41′ is through slot formed at the surrounding wall 12′ of the collector 10′ at the lower portion thereof to communicate with the urine guider 30′. Preferably, the discharging unit 40′ further comprises a discharging valve 43′ coupled at the discharging port 41′, such that the discharging valve 43′ is selectively opened for discharging urine from the urine guider 30′ and closed for retaining urine at the urine guider 30′. It is worth mentioning that the discharging port 41′ is formed at the surrounding wall 12′ of the collector 10′, such that the bottom wall 11′ of the collector 10′ can be stably placed on a supporting surface, such as bed surface.

The discharging unit 40′ further comprises a container 44′ detachably linked to the transporting tube 42′ for collecting urine from the urine guider 30′ through the transporting tube 42′. Accordingly, the transporting tube 42′ has one end detachably coupled at the discharging port 41′ and an opposed end detachably coupled to the container 44′. Therefore, once the discharging valve 43′ is opened, the urine at the urine guider 30′ will be discharged to the container 44′ through the transporting tube 42′. It is worth mentioning that the transporting tube 42′ can be extended to a drain outlet to directly discharge the urine to the drain outlet without the container 44′.

In order to use the urine collector of the present invention, the user is able to place the basin under the hip of the user, wherein the user is able to sit on top of the basin to align the urethra of the user within the top opening of the basin. As a result, the urine will be temporary stored at the urine guider 30′ and will then be guided to discharge to the container 44′. Depending the volume of the container 44′, the user is able to re-use the basin for a relatively long period of time, such as overnight, without directly draining the urine out of the basin manually, so as to reduce the workload of the healthcare worker or family member. It is worth mentioning that the urine collector can be cleaned by directly pouring clean water into the cavity of the collector 10′ through the top opening to rinse the entire urine collector and discharging the water from the collector 10′ to the container 30′.

According to the above mentioned embodiments and their alternatives, the collector 1, 10′ can be made of polymer materials, such as rigid plastic, rigid rubber, or polycarbonate materials. In other words, since the collector 1, 10′ is made of light in weight materials, the collector 1, 10′ is easy to be carried, and it is convenient for the patients to use. In addition, the transporting tube 2, 42′ of the embodiments and their alternatives can be made of PET, silica gel, and latex, such that the above mentioned materials are light in weight and are durable.

In view of above embodiments, the urine collector of the present invention is constructed in a compact structure that has a relatively thinner structure and a light weight, such that the urine collector can discharge urine continuously and be used by the patients for overnight. In other words, health care workers do not need to frequently unload the urine in the urine collector of the present invention.

In addition, the urine collector of the present invention is adapted to collect urine throughout the hip area but not limiting to the urination portion, so as to prevent leaking out of urine. Also, a top side a top cover of the collector of the urine collector is inclinedly extended from an outer peripheral edge toward a center, so as to ensure and guide all the urine be collected toward a container through a top opening of the collector to avoid splashing and leakage. Since the collector is not used to store urine being collected but to function as a transition means to continue to guide the container, the collector can be constructed in compact size with a thinner thickness and a lighter weight that enables an overnight usage without the need to replace the collector. Accordingly, to a less severe patient, the patient can place the collector in position by himself or herself and there is no need to have anyone to help for urination. To a more severe patient, it simply needs someone to slight support the hip of the patient to place the collector in position for urination that substantially alleviates suffering to the patient and reduces burden of the care for the patient. For hospitals or senior care facilities, the collector can be directly connected to the sewage drain system that links to a urine collection container for ease of filtration or disinfection treatment, so that the present invention can save a lot of manpower and meet the environmental health requirement.

It is worth mentioning that the urine collector of the present invention is suitable for all kinds of people to collect urine in bed, especially for post-partum women, seniors, people with problems of frequent urination or urinary incontinence, and injured and sick patients. It is a urine collector enabling woman in bed to urinate without leakage and avoiding overflowing of urine. The present invention provides a simple structure and compact size that can be manufactured by inexpensive material and in low cost, especially good for mass production without using complicated and expensive production tools and machines, benefiting both the consumers and the manufacturers.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A urine collector for collecting urine from a user, comprising:
a basin having a cavity, a top side having a size adapted for covering a hip of the user, and a top opening formed at said top side to communicate with said cavity; and
a urine guider provided at a bottom portion of said basin for collecting urine at said urine guider within said cavity of said basin.

2. The urine collector, as recited in claim 1, wherein said basin comprises a collector defining said cavity therein, and a top cover detachably supported on top of said collector, wherein said top cover has a ring shape defining an outer peripheral edge, an to inner peripheral edge, and a through hole within said inner peripheral edge to form said top opening of said basin for communicating with said cavity, wherein said top side of said basin is defined at a top side of said top cover for ensuring urine flowing into said cavity through said through hole.

3. The urine collector, as recited in claim 2, wherein said urine guider provided at a bottom wall of said collector at a peripheral portion thereof for collecting the urine at said urine guider within said cavity of said basin so as to enhance a stabilization of said collector after the urine is collected.

4. The urine collector, as recited in claim 2, wherein said top side of said top cover is sloped downwardly from said outer peripheral edge of said top cover to said inner peripheral edge thereof for guiding the urine to flow into said collector.

5. The urine collector, as recited in claim 3, wherein said top side of said top cover is sloped downwardly from said outer peripheral edge of said top cover to said inner peripheral edge thereof for guiding the urine to flow into said collector.

6. The urine collector, as recited in claim 2, wherein said top cover further has a plurality of urine directing grooves indently formed on said top side of said top cover and extended to said inner peripheral edge thereof for guiding the urine to flow toward said through hole of said top cover.

7. The urine collector, as recited in claim 5, wherein said top cover further has a plurality of urine directing grooves indently formed on said top side of said top cover and extended to said inner peripheral edge thereof for guiding the urine to flow toward said through hole of said top cover.

8. The urine collector, as recited in claim 1, further comprising a discharging unit operatively connected with said urine guider, wherein said discharging unit comprises a discharging port formed at said basin to communicate with said urine guider and a transporting tube detachably coupled at said discharging port for discharging the urine at said urine guider through said discharging port.

9. The urine collector, as recited in claim 5, further comprising a discharging unit operatively connected with said urine guider, wherein said discharging unit comprises a discharging port formed at said basin to communicate with said urine guider and a transporting tube detachably coupled at said discharging port for discharging the urine at said urine guider through said discharging port.

10. The urine collector, as recited in claim 7, further comprising a discharging unit operatively connected with said urine guider, wherein said discharging unit comprises a discharging port formed at said basin to communicate with said urine guider and a transporting tube detachably coupled at said discharging port for discharging the urine at said urine guider through said discharging port.

11. The urine collector, as recited in claim 8, wherein said discharging unit further comprises a container detachably linked to said transporting tube for collecting urine from said urine guider through said transporting tube.

12. The urine collector, as recited in claim 9, wherein said discharging unit further comprises a container detachably linked to said transporting tube for collecting urine from said urine guider through said transporting tube.

13. The urine collector, as recited in claim 10, wherein said discharging unit further comprises a container detachably linked to said transporting tube for collecting urine from said urine guider through said transporting tube.

14. The urine collector, as recited in claim 3, wherein said urine guider has a guiding groove indentedly formed at said peripheral portion of said bottom wall of said collector for collecting the urine at said peripheral portion of said collector so as to enhance a stabilization of said collector after the urine is collected.

15. The urine collector, as recited in claim 13, wherein said urine guider has a guiding groove indentedly formed at said peripheral portion of said bottom wall of said collector for collecting the urine at said peripheral portion of said collector so as to enhance a stabilization of said collector after the urine is collected.

16. The urine collector, as recited in claim 3, wherein said urine guider is integrated with said bottom wall of said container that said bottom wall is a convex wall radially sloping down toward said peripheral portion for collecting the urine at said peripheral portion of said collector so as to enhance a stabilization of said collector after the urine is collected.

17. The urine collector, as recited in claim 13, wherein said urine guider is integrated with said bottom wall of said container that said bottom wall is a convex wall radially sloping down toward a peripheral portion for collecting the urine at said peripheral portion of said collector so as to enhance a stabilization of said collector after the urine is collected.

18. The urine collector, as recited in claim 10, wherein said collector further has a surrounding wall upwardly extended from said bottom wall to define said cavity within said surrounding wall and said bottom wall, wherein said discharging port is formed at said surrounding wall of said container to communicate with said urine guider.

19. The urine collector, as recited in claim 15, wherein said collector further has a surrounding wall upwardly extended from said bottom wall to define said cavity within said surrounding wall and said bottom wall, wherein said discharging port is formed at said surrounding wall of said container to communicate with said urine guider.

20. The urine collector, as recited in claim 17, wherein said collector further has a surrounding wall upwardly extended from said bottom wall to define said cavity within said surrounding wall and said bottom wall, wherein said discharging port is formed at said surrounding wall of said container to communicate with said urine guider.
